(19) United States
(12) Patent Application Publication
(10) Pub. No.: US 2024/0424094 A1
(43) Pub. Date: Dec. 26, 2024

(54) METHODS AND COMPOSITIONS FOR CANCER TREATMENT

(71) Applicant: CancerVAX, Inc., Santa Barbara, CA (US)

(72) Inventors: Ryan Davies, Santa Barbara, CA (US); Jonathan Lei, Santa Barbara, CA (US)

(73) Assignee: CancerVAX Inc., Santa Barbara, CA (US)

(21) Appl. No.: 18/698,262

(22) PCT Filed: Oct. 4, 2022
(86) PCT No.: PCT/US2022/045632

§ 371 Date: Apr. 3, 2024

Related U.S. Application Data
(60) Provisional application No. 63/252,890, filed on Oct. 6, 2021.

(51) Int. Cl. A61K 39/395; A61K 31/7088; A61K 38/46; A61K 39/00; A61P 37/04

(52) U.S. Cl. CPC A61K 39/39558; A61K 31/7088; A61K 38/465; A61K 39/0011; A61P 37/04

(57) ABSTRACT

Nucleic acid mutation associated with specific cancers can be targeted using a CRISPR detector with a special mRNA pay load containing instructions for the target cell to express a universal cancer antigen.

### CLAIM FOR PRIORITY

This application claims priority under 35 U.S.C. § 119(e) to U.S. Patent Application Ser. No. 63/252,890, filed on Oct. 6, 2021, which is hereby incorporated by reference in its entirety.

### FIELD OF THE INVENTION

The invention relates to a novel vaccine platform for treating cancer.

### BACKGROUND

Cancer treatment vaccines generally attempt to mount an attack against cancer cells in the body by involving the immune system. Some cancer treatment vaccines are made up of cancer cells, parts of cells, or pure antigens (certain proteins on the cancer cells). Sometimes a patient's own immune cells are removed and exposed to these substances in the lab to create the vaccine. The vaccine is injected into the body to increase or improve the immune response against cancer cells. While there are a wide range of cancer therapies, there is a need for a cancer treatment that is specific, simple to administer and effective. Antibodies are unique in their ability to both directly kill tumor cells while simultaneously engage the host immune system to develop long-lasting effector responses against the tumor. The combination of a multifaceted mechanism of action with target specificity distinguishes mAb therapy from treatments such as chemotherapy and underlies the capability of antibodies to elicit strong anti-tumor responses while minimizing toxicity and adverse events.

### SUMMARY OF THE INVENTION

Using the claimed platform, a nucleic acid mutation associated with specific cancers can be targeted using a CRISPR detector with a special mRNA payload containing instructions for the target cell to express a universal cancer antigen (UCA). A unique detector can be designed for a specific type or category of cancers. Computer-based artificial intelligence or machine learning can be used to identify of reliable mutation markers.

In general, a method for targeting cancer-associated mutations can include identifying a nucleic acid mutation associated with a specific cancer, detecting the nucleic acid mutation using a CRISPR detector and an mRNA payload, upon detection, releasing the mRNA payload into a target cell, the mRNA payload containing instructions for the target cell to express a universal cancer antigen (UCA), allowing the target cell to express a universal cancer antigen; and applying a UCA antibody designed to target those cells with UCA.

In certain embodiments, the CRISPR detector and mRNA payload are provided together in a single shot. In certain embodiments, the CRISPR detector and mRNA payload result in expression of a universal cancer antigen that is targeted by a subject's existing antibodies. In certain embodiments, the universal cancer antigen is targeted by an antibody provided in a vaccine. In certain embodiments, the CRISPR detector and mRNA payload are provided in separate shots. In certain embodiments, the shots are provided simultaneously. In other embodiments, the shots are provided sequentially.

In certain embodiments, the UCA can be a non-naturally occurring protein.

The nucleic acid can be DNA. The nucleic acid can be RNA.

In certain embodiments, the antibody can induce cell death of the target cell.

In certain embodiments, the antibody can reduce tumorigenesis.

In certain embodiments, the antibody can reduce angiogenesis.

In certain embodiments, the antibody can activate an immune response.

In certain embodiments, the antibody can activate an effector cell response.

In certain embodiments, the antibody can activate a memory cell response.

In certain embodiments, the antibody can activate modulate a cytokine response.

In certain embodiments, the antibody can modulate an interferon response.

In certain embodiments, the antibody can modulate an interleukin response.

In certain embodiments, the antibody can be a bi-specific antibody that binds T-cells to ensure a more precise immune response.

In certain embodiments, the method can further include administering an adjuvant.

In general, a composition for treating cancer can include a CRISPR detector, an mRNA payload containing instructions for the tumor cell to express a universal cancer (UCA) antigen and a UCA antibody designed to target those cells with UCA expression.

In certain embodiments, a method for targeting cancer-associated mutations can include identifying a nucleic acid mutation associated with a specific cancer, detecting the nucleic acid mutation using a CRISPR detector and a payload containing genetic material, upon detection, releasing the payload into a target cell, the payload containing instructions for the target cell to express a universal cancer antigen (UCA), allowing the target cell to express a universal cancer antigen; and allowing a UCA antibody designed to target those cells expressing the UCA.

In certain embodiments, the genetic material can be a non-replicating mRNA.

In certain embodiments, the genetic material can be a virally derived mRNA.

In certain embodiments, the genetic material can be self-amplifying RNA.

### BRIEF DESCRIPTION OF THE DRAWINGS

FIG. 1 depicts one embodiment of the cancer vaccine platform in which the mRNA contains instructions for a target cell to express a UCA.

FIG. 2 depicts another embodiment of the cancer vaccine platform in which the mRNA contains instructions for an existing vaccine antigen.

FIG. 3 depicts a one-shot embodiment of the cancer vaccine platform.

FIG. 4 depicts an embodiment of the cancer vaccine platform in which the CRISPR detector and mRNA payload are provided in a single shot prior to a second vaccine shot.

FIG. 5 depicts an embodiment of the cancer vaccine platform in which the CRISPR detector and mRNA payload are provided in a separate shots prior to a later vaccine shot such that there are three separate shots with unique compositions.

### DETAILED DESCRIPTION OF THE INVENTION

Vaccines generally fall into four different types: whole-pathogen vaccines, subunit vaccines, and nucleic acid vaccines. Nucleic acid vaccines are the most recent innovation; they include plasmid DNA vaccines and mRNA vaccines. Each approach uses genetic material that encodes one or more antigenic proteins. Upon vaccination, the genetic payload enters the cytosol, or liquid matrix, of human cells, where the cellular machinery uses the genetic material to produce antigens that elicit an immune response. A payload refers to an amount of antigen (e.g. genetic material, protein or lipid) that elicits an immune response. See, e.g. Lowe, D., RNA Vaccines And Their Lipids, www.science.org/content/blog-post/rna-vaccines-and-their-lipids (January 2021). As vaccine platforms, nucleic acid technologies have a number of distinct advantages over older approaches. First, they are fast and easy to manufacture. Second, the encoded immunogenic proteins do not remain in the human body for very long. Third, the immune system amplifies the genetic material in response even to small amounts of the expressed antigenic protein. A vaccine payload that contains an information-coding molecule, such as RNA, is revolutionary because what the immune system responds to is not the information coding molecule (e.g., RNA), but something the information coding molecule teaches the subject cells to make.

The amount of target gene modulation may be measured by any suitable method known in the art. In some embodiments, the “effective amount” or “therapeutically effective amount” is the amount of a composition that is required to ameliorate the symptoms of a disease relative to an untreated patient. In some embodiments, an effective amount is the amount of a composition sufficient to introduce an alteration in a gene of interest in a cell (e.g., a cell in vitro, ex vivo or in vivo).

### Mutations and Cancer

Mutations are changes in the genetic sequence, and they are a main cause of diversity among organisms. These changes occur at many different levels, and they can have widely differing consequences. Although various types of molecular changes exist, the word “mutation” typically refers to a change that affects the nucleic acids. In cellular organisms, these nucleic acids are the building blocks of DNA, and in viruses they are the building blocks of either DNA or RNA. The smallest mutations are point mutations, in which only a single base pair is changed into another base pair. Yet another type of mutation is the nonsynonymous mutation, in which an amino acid sequence is changed. Such mutations lead to either the production of a different protein or the premature termination of a protein. As opposed to nonsynonymous mutations, synonymous mutations do not change an amino acid sequence, although they occur, by definition, only in sequences that code for amino acids. Synonymous mutations exist because many amino acids are encoded by multiple codons. Base pairs can also have diverse regulating properties if they are located in introns, intergenic regions, or even within the coding sequence of genes. For some historic reasons, all of these groups are often subsumed with synonymous mutations under the label “silent” mutations. Depending on their function, such silent mutations can be anything from truly silent to extraordinarily important, the latter implying that working sequences are kept constant by purifying selection. Loewe, L. (2008) Genetic mutation. Nature Education 1 (1):113.

Mutations may also take the form of insertions or deletions, which are together known as indels. Indels can have a wide variety of lengths. At the short end of the spectrum, indels of one or two base pairs within coding sequences have the greatest effect, because they will inevitably cause a frameshift (only the addition of one or more three-base-pair codons will keep a protein approximately intact). At the intermediate level, indels can affect parts of a gene or whole groups of genes. At the largest level, whole chromosomes or even whole copies of the genome can be affected by insertions or deletions, although such mutations are usually no longer subsumed under the label indel. At this high level, it is also possible to invert or translocate entire sections of a chromosome, and chromosomes can even fuse or break apart. If a large number of genes are lost as a result of one of these processes, then the consequences are usually very harmful. However, different genetic systems react differently to such events. Finally, still other sources of mutations are the many different types of transposable elements, which are small entities of DNA that possess a mechanism that permits them to move around within the genome. Some of these elements copy and paste themselves into new locations, while others use a cut-and-paste method. Such movements can disrupt existing gene functions (by insertion in the middle of another gene), activate dormant gene functions (by perfect excision from a gene that was switched off by an earlier insertion), or occasionally lead to the production of new genes (by pasting material from different genes together).

Some genes control cell division. When mutations occur in these genes, a cell may begin to divide without control. Cells that divide when they are not supposed to eventually lead to cancer. All cancer is the result of gene mutations. Mutations may be caused by aging, exposure to chemicals, radiation, hormones or other factors in the body and the environment. Over time, a number of mutations may occur in a single cell, allowing it to divide and grow in a way that becomes a cancer. This usually takes many years, and explains why most cancers occur at a later age in life. Because most people are not born with these “acquired” gene mutations, they cannot pass them on to their children.

Cancers can be categorized into two groups: those whose frequency increases with age, and those resulting from errors during mammalian development. The first group is linked to DNA replication through the accumulation of genetic mutations that occur during proliferation of developmentally acquired stem cells that give rise to and maintain tissues and organs. These mutations, which result from DNA replication errors as well as environmental insults, fall into two categories; cancer driver mutations that initiate carcinogenesis and genome destabilizing mutations that promote aneuploidy through excess genome duplication and chromatid missegregation. Increased genome instability results in accelerated clonal evolution leading to the appearance of more aggressive clones with increased drug resistance. The second group of cancers, termed germ cell neoplasia, results from the mislocation of pluripotent stem cells during early development. During normal development, pluripotent stem cells that originate in early embryos give rise to all of the cell lineages in the embryo and adult, but when they mislocate to ectopic sites, they produce tumors. Remarkably, pluripotent stem cells, like many cancer cells, depend on the Geminin protein to prevent excess DNA replication from triggering DNA damage-dependent apoptosis. This link between the control of DNA replication during early development and germ cell neoplasia reveals Geminin as a potential chemotherapeutic target in the eradication of cancer progenitor cells.

There are 4 main types of genes involved in cell division: oncogenes, tumor suppressor genes, DNA repair genes, and self-destruction genes. See, e.g., cancerresearchuk.org/about-cancer/what-is-cancer/genes-dna-and-cancer. Most tumors have faulty copies of more than 1 of these types. If we can reliably detect these faulty genes, then we can then target the tumor with an immunotherapy vaccine.

Because cancer, in essence, escapes the body's normal immune surveillance process, a novel method of cancer treatment can be premised on allowing the cancer to look or act more suspicious to the immune system, and helping the immune system to “learn” how to identify the cancer. For certain cancer patients both have an overproduction of cytokines, which makes the immune system more vulnerable. There are other relationships between cytokine responses (e.g., with COVID-19 and Ewing sarcoma specifically) that can be used to customize a novel vaccine. Specifically, one can isolate certain cells from a sarcoma patient, expose those cells to different protein conjugates for future treatment. Similarly, knowing that Ewing tumors are usually driven by a well-known hallmark of gene fusion involving the Ewing Sarcoma Breakpoint Region 1 (EWSR1) gene, one can control fusion gene expression to result in the production of a specific protein as a universal cancer antigen, and use this as a hallmark that can be targeted by antibodies designed to attack this protein.

### mRNA Vaccines

Messenger RNA is a type of RNA that is necessary for protein production. mRNA is the intermediate step between the translation of protein-encoding DNA and the production of proteins by ribosomes in the cytoplasm. Two major types of RNA are currently studied as vaccines: non-replicating mRNA and virally derived, self-amplifying RNA. Conventional mRNA-based vaccines encode the antigen of interest and contain 5′ and 3′ untranslated regions (UTRs), whereas self-amplifying RNAs encode not only the antigen but also the viral replication machinery that enables intracellular RNA amplification and abundant protein expression. In cells, mRNA uses the information in genes to create a blueprint for making proteins. Once cells finish making a protein, the mRNA is broken down. mRNA from vaccines does not enter the nucleus and does not alter DNA. In general mRNA molecules contain the genetic material that provide instructions for our body on how to make a viral protein that triggers an immune response within our bodies.

mRNA vaccines represent a promising alternative to conventional vaccine approaches because of their high potency, capacity for rapid development and potential for low-cost manufacture and safe administration. However, their application has until recently been restricted by the instability and inefficient in vivo delivery of mRNA. Recent technological advances have now largely overcome these issues, and multiple mRNA vaccine platforms against infectious diseases and several types of cancer have demonstrated encouraging results in both animal models and humans. Pardi, N., et al. mRNA vaccines—a new era in vaccinology. Nat Rev Drug Discov 17, 261-279 (2018). Preclinical studies have created hope that mRNA vaccines will fulfil many aspects of an ideal clinical vaccine: they have shown a favourable safety profile in animals, are versatile and rapid to design for emerging infectious diseases, and are amenable to scalable good manufacturing practice (GMP) production (already under way by several companies).

Unlike protein immunization, several formats of mRNA vaccines induce strong CD8+ T cell responses, likely owing to the efficient presentation of endogenously produced antigens on MHC class I molecules, in addition to potent CD4+ T cell responses. Additionally, unlike DNA immunization, mRNA vaccines have shown the ability to generate potent neutralizing antibody responses in animals with only one or two low-dose immunizations. As a result, mRNA vaccines have elicited protective immunity against a variety of infectious agents in animal models. Id. Two major types of RNA vaccine have been utilized against infectious pathogens: self-amplifying or replicon RNA vaccines and non-replicating mRNA vaccines. Non-replicating mRNA vaccines can be further distinguished by their delivery method: ex vivo loading of DCs or direct in vivo injection into a variety of anatomical sites.

The 5′ and 3′ UTR elements flanking the coding sequence profoundly influence the stability and translation of mRNA, both of which are critical concerns for vaccines. These regulatory sequences can be derived from viral or eukaryotic genes and greatly increase the half-life and expression of therapeutic mRNAs. See Pardi, N., et al. mRNA vaccines—a new era in vaccinology. Nat Rev Drug Discov 17, 261-279 (2018). A 5′ cap structure is required for efficient protein production from mRNA Gallie, D. R. The cap and poly(A) tail function synergistically to regulate mRNA translational efficiency. Genes Dev. 5, 2108-2116 (1991). Various versions of 5′ caps can be added during or after the transcription reaction using a vaccinia virus capping enzyme (Martin, S. A., Paoletti, E. & Moss, B. Purification of mRNA guanylyltransferase and mRNA (guanine-7-) methyltransferase from vaccinia virions. J. Biol. Chem. 250, 9322-9329 (1975)) or by incorporating synthetic cap or anti-reverse cap analogues (Stepinski, J., Waddell, C., Stolarski, R., Darzynkiewicz, E. & Rhoads, R. E. Synthesis and properties of mRNAs containing the novel “anti-reverse” cap analogs 7-methyl(3′-O-methyl)GpppG and 7-methyl(3′-deoxy)GpppG. RNA 7, 1486-1495 (2001); Malone, R. W., Felgner, P. L. & Verma, I. M. Cationic liposome-mediated RNA transfection. Proc. Natl Acad. Sci. USA 86, 6077-6081 (1989).

The poly(A) tail also plays an important regulatory role in mRNA translation and stability; thus, an optimal length of poly(A) must be added to mRNA either directly from the encoding DNA template or by using poly(A) polymerase. Holtkamp, S. et al. Modification of antigen-encoding RNA increases stability, translational efficacy, and T-cell stimulatory capacity of dendritic cells. Blood 108, 4009-4017 (2006). The codon usage additionally has an impact on protein translation. Replacing rare codons with frequently used synonymous codons that have abundant cognate tRNA in the cytosol is a common practice to increase protein production from mRNA, although the accuracy of this model has been questioned. Gustafsson, C., Govindarajan, S. & Minshull, J. Codon bias and heterologous protein expression. Trends Biotechnol. 22, 346-353 (2004); Mauro, V. P. & Chappell, S. A. A critical analysis of codon optimization in human therapeutics. Trends Mol. Med. 20, 604-613 (2014). Enrichment of G:C content constitutes another form of sequence optimization that has been shown to increase steady-state mRNA levels in vitro and protein expression in vivo. Kudla, G., Lipinski, L., Caffin, F., Helwak, A. & Zylicz, M. High guanine and cytosine content increases mRNA levels in mammalian cells. PLoS Biol. 4, e180 (2006); Thess, A. et al. Sequence-engineered mRNA without chemical nucleoside modifications enables an effective protein therapy in large animals. Mol. Ther. 23, 1456-1464 (2015). Although protein expression may be positively modulated by altering the codon composition or by introducing modified nucleosides, it is also possible that these forms of sequence engineering could affect mRNA secondary structure, the kinetics and accuracy of translation and simultaneous protein folding, and the expression of cryptic T cell epitopes present in alternative reading frames. All these factors could potentially influence the magnitude or specificity of the immune response. Id.

The production of in vitro transcribed (IVT) mRNA can be carried out in cell-free systems, leading to easy standardization of clinical-grade manufacturing, which can be performed under Good Manufacturing Practices (GMPs). Fabrication costs of IVT mRNA under GMPs are substantially low as compared to recombinant proteins produced in eukaryotic cells. It is important to select an efficient purification method of the IVT mRNA in order to eliminate aberrant (e.g., truncated) mRNA molecules, which are highly immunogenic contaminants and may lower translation efficiency. Manufacturing of IVT mRNA by a cell-free in vitro transcription system requires a linearized DNA template which must contain a prokaryotic phage promoter sequence for the T3, T7, or SP6 RNA polymerases, the open reading frame (ORF) encoding the desired protein, the sequences corresponding to the regulatory untranslated regions (UTRs), and optionally, to a polyadenylated tail (poly(A) tail). When the poly(A) tail is not encoded directly in the DNA template, it can be added post-transcriptionally by enzymatic reactions with recombinant poly(A) polymerase of E. coli (E-PAP). Since the final IVT mRNA must be structurally similar to natural mRNA processed in the cytoplasm of eukaryotic cells, it also needs to be capped in 5′. A synthetic IVT mRNA consists of the following five fundamental structures, which can be chemically modified in order to optimize the translation process and the stability, and to regulate the immunogenicity: (a) Cap in 5′; (b) 5′ UTR; (c) an ORF, which has the starting codon AUG and the stop codon (UAA, UAG, UGA); (d) 3′ UTR; and (e) poly(A) tail. Chemical modifications influence in a specific manner the mRNA translation in different cell types. Therefore, addressing the precise intracellular behavior of mRNA in the cell of interest will lead to further chemical modifications and extend the usefulness of mRNA as a biomedical product.

### Internalization of mRNA

The first step for efficient internalization of in vitro transcribed (IVT) mRNA can be the interaction between the delivery system and the cell membrane. The attachment to the cell surface may occur through electrostatic interactions between the system and the membrane surface, which is favored for those systems presenting a cationic nature. Cell binding can also be improved by incorporating ligands able to interact with specific cell surface receptors into the vectors. The main mechanism of cell entry is endocytosis. It comprises a variety of complex processes that determine the intracellular disposition of the mRNA. The vectors are included in endosomes by the invagination of the cell membrane. Endosomes mature and fuse with lysosomes, where the acidic environment and the presence of hydrolytic enzymes can degrade the vector and the nucleic acid. Therefore, endosomal escape before degradation is considered a bottleneck for successful mRNA therapy, and, as in the case of cellular internalization, the delivery system plays a crucial role. The foremost proposed mechanisms of endosomal escape include endosome disruption, active transport, or fusion of the delivery system with the endosomal membrane. However, it was recently identified that late endosome/lysosome formation is essential for the functional delivery of exogenously presented mRNA.

mRNA vaccines work by introducing a piece of mRNA that corresponds to a viral protein, usually a small piece of a protein found on the virus's outer membrane. Individuals who get an mRNA vaccine are not exposed to the virus, nor can they become infected by the vaccine. Using this mRNA blueprint, cells produce the viral protein.

As part of a normal immune response, the immune system recognizes that the protein is foreign and produces specialized proteins called antibodies. Antibodies help protect the body against infection by recognizing individual viruses or other pathogens, attaching to them, and marking the pathogens for destruction. Once produced, antibodies remain in the body, even after the body has rid itself of the pathogen, so that the immune system can quickly respond if exposed again. If a person is exposed to a virus after receiving mRNA vaccination for it, antibodies can quickly recognize it, attach to it, and mark it for destruction before it can cause serious illness

### Modulation of Immunogenicity

Exogenous mRNA is inherently immunostimulatory, as it is recognized by a variety of cell surface, endosomal and cytosolic innate immune receptors. Depending on the therapeutic application, this feature of mRNA could be beneficial or detrimental. It is potentially advantageous for vaccination because in some cases it may provide adjuvant activity to drive dendritic cell (DC) maturation and thus elicit robust T and B cell immune responses. However, innate immune sensing of mRNA has also been associated with the inhibition of antigen expression and may negatively affect the immune response. Kariko, K. et al. Incorporation of pseudouridine into mRNA yields superior nonimmunogenic vector with increased translational capacity and biological stability. Mol. Ther. 16, 1833-1840 (2008); Kariko, K., Muramatsu, H., Ludwig, J. & Weissman, D. Generating the optimal mRNA for therapy: HPLC purification eliminates immune activation and improves translation of nucleoside-modified, protein-encoding mRNA. Nucleic Acids Res. 39, c142 (2011). Although the paradoxical effects of innate immune sensing on different formats of mRNA vaccines are incompletely understood, some progress has been made in recent years in elucidating these phenomena. Studies over the past decade have shown that the immunostimulatory profile of mRNA can be shaped by the purification of IVT mRNA and the introduction of modified nucleosides as well as by complexing the mRNA with various carrier molecules. See, e.g., Kariko (2008), Kariko (2011), Fotin-Mleczek, M. et al. Messenger RNA-based vaccines with dual activity induce balanced TLR-7 dependent adaptive immune responses and provide antitumor activity. J. Immunother. 34, 1-15 (2011). As a mimic of viral genomes and replication intermediates, dsRNA is a potent pathogen-associated molecular pattern (PAMP) that is sensed by pattern recognition receptors in multiple cellular compartments. Recognition of IVT mRNA contaminated with dsRNA results in robust type I interferon production, which upregulates the expression and activation of protein kinase R (PKR; also known as EIF2AK2) and 2′-5′-oligoadenylate synthetase (OAS), leading to the inhibition of translation and the degradation of cellular mRNA and ribosomal RNA, respectively. Studies have demonstrated that contaminating dsRNA can be efficiently removed from IVT mRNA by chromatographic methods such as reverse-phase fast protein liquid chromatography (FPLC) or high-performance liquid chromatography (HPLC). Strikingly, purification by FPLC has been shown to increase protein production from IVT mRNA by up to 1,000-fold in primary human DCs. Thus, appropriate purification of IVT mRNA seems to be critical for maximizing protein (immunogen) production in DCs and for avoiding unwanted innate immune activation.

Besides dsRNA contaminants, single-stranded mRNA molecules are themselves a PAMP when delivered to cells exogenously. Single-stranded oligoribonucleotides and their degradative products are detected by the endosomal sensors Toll-like receptor 7 (TLR7) and TLR8, resulting in type I interferon production. Crucially, it was discovered that the incorporation of naturally occurring chemically modified nucleosides, including but not limited to pseudouridine and 1-methylpseudouridine, prevents activation of TLR7, TLR8 and other innate immune sensors, thus reducing type I interferon signalling. Nucleoside modification also partially suppresses the recognition of dsRNA species. Anderson, B. R. et al. Incorporation of pseudouridine into mRNA enhances translation by diminishing PKR activation. Nucleic Acids Res. 38, 5884-5892 (2010). As a result, nucleoside-modified mRNA is translated more efficiently than unmodified mRNA in vitro, particularly in primary DCs, and in vivo in mice. Notably, the highest level of protein production in DCs was observed when mRNA was both FPLC-purified and nucleoside-modified. These advances in understanding the sources of innate immune sensing and how to avoid their adverse effects have substantially contributed to the current interest in mRNA-based vaccines and protein replacement therapies. Pardi, N., et al. mRNA vaccines—a new era in vaccinology. Nat Rev Drug Discov 17, 261-279 (2018).

### Novel Cancer Vaccine Platform

Cancer vaccines cause the immune system to attack cells with one or more specific antigens. Because the immune system has special cells for memory, the vaccine platform is designed and expected to work long after it is given. The inventors have discovered a platform that can be applied by using a CRISPR detector with a specific mRNA payload that contain instructions for the tumor cells to express a custom Universal Cancer Antigen (UCA). The novel platform has three components: detection, marking, and vaccination.

### CRISPR Detection

Using CRISPR to treat most people with genetic disorders requires clearing an enormous hurdle: getting the molecular scissors into the body and having it slice DNA in the tissues where it's needed. Recently, researchers have injected a CRISPR drug into the blood of people born with a disease that causes fatal nerve and heart disease and shown that in three of them it nearly shut off production of toxic protein by their livers. Thus, CRISPR has proven to work therapeutically inside the body. Sec, e.g., J. Kaier, CRISPR injected into the blood treats a genetic disease for first time, Science, June 2021, www.science.org/news/2021/06/crispr-injected-blood-treats-genetic-disease-first-time.

Because of the need for precise diagnosis in many disease situations, another important application of the CRISPR-Cas system has emerged: detection of diseases and microbes. Although robust and potent platforms were developed based on CRISPR-Cas9, the discovery of Cas13a (formerly C2c2) and Cas12a (formerly Cpf1) which both have collateral cleavage activity has revolutionized the field of nucleic acid detection. Cas13a is a single-component, RNA-guided and targeting enzyme, which is specific for ssRNA and collaterally cleaves neighbor non-targeted RNAs. In contrast, Cas12a is an RNA-guided, DNA-targeting enzyme which targets DNA and collaterally cleaves ssDNA. Different platforms have been developed based on these two proteins. Specific high sensitivity enzymatic reporter unlocking (SHERLOCK) was introduced by Gootenberg in 2017, which exploits Cas13a for the detection of RNA molecules and a diagnostic platform based on this method was developed in 2018 At the same time, a Cas-12a-based diagnostic tool called one-hour low-cost multipurpose highly efficient system (HOLMES) was introduced in 2017 and a diagnosis platform was described in 2018. A striking example of the power of these systems is the extremely fast development of a CRISPR-Cas-based diagnostic test, which can rapidly and with a high sensitivity diagnose SARS-CoV-2, an emerging virus responsible for the COVID-19 pneumonia disease. This demonstrated the high potential of CRISPR-Cas systems for the development of rapid detection of newly emerging diseases. Jolany vangah, S., Katalani, C., Boone, H.A. et al. CRISPR-Based Diagnosis of Infectious and Noninfectious Diseases. Biol Proced Online 22, 22 (2020). https://doi.org/10.1186/s12575-020-00135-3.

Detection can be performed to identify DNA/RNA based mutation for specific cancers using a CRISPR detector with a special mRNA payload. A different detector is designed for each cancer or group of cancers. Computer-based AI or machine learning can be used in the discovery of reliable mutation markers. The ICGC/TCGA Pan-Cancer Analysis of Whole Genomes Project (PCAWG, or the Pan-Cancer Project), a collaboration involving more than 1,300 scientists and clinicians from 37 countries, analyzed more than 2,600 whole genomes of 38 different tumor types-the largest publicly available whole-genome dataset in the cancer genomics field. Fifty-two members of the Broad Institute of MIT and Harvard contributed to this research throughout the six-year long project. Using the collected data, 16 working groups examined multiple aspects of cancer development, causation, progression, and classification, confirming previous findings and generating new knowledge about cancer biology, including identifying a large diversity of molecular processes that generate cancer-causing mutations. The Pan-Cancer Project also improved and developed new methods for analyzing cancer genomes. Previous cancer genome studies focused on the 1 percent of the genome that codes for proteins, known as the exome. The Pan-Cancer Project explored the remaining 99 percent of the genome, which includes regions that regulate the activity of genes. See, S. McPherson, Collaboration generates most complete cancer genome map, The Harvard Gazette, Feb. 5, 2020, news.harvard.edu/gazette/story/2020/02/big-step-toward-identifying-all-cancer-causing-genetic-mutations/.

CRISPR/Cas systems have been used for genome editing, based on their ability to accurately recognize and cleave specific DNA and RNA sequences. Moreover, following recognition of the target sequence, certain CRISPR/Cas systems including orthologues of Cas13, Cas12a, and Cas14 exhibit collateral nonspecific catalytic activities that can be employed for nucleic acid detection, for example by degradation of a labeled nucleic acid to produce a fluorescent signal. CRISPR/Cas systems are amenable to multiplexing, thereby enabling a single diagnostic test to identify multiple targets down to attomolar (10-18 mol/L) concentrations of target molecules. Developing devices that couple CRISPR/Cas with lateral flow systems allow inexpensive, accurate, highly sensitive, in-field deployable diagnostics. These sensors have myriad applications, from human health to agriculture. CRISPR-based biosensing technologies have significant potential use in a myriad of applications. See, R. Aman, et al., ACS Synth. Biol. 2020, 9, 6, 1226-1233, Publication Date: Mar. 11, 2020, https://doi.org/10.1021/acssynbio.9b00507.

The CRISPR detector can refer to the polypeptide or polypeptide components involved in gene editing, or any polynucleotide(s) encoding the polypeptide or polypeptide components. In some embodiments, the polypeptide domain having DNA binding activity is a polypeptide domain having programmable DNA binding activity. In some embodiments, the polypeptide domain having programmable DNA binding activity comprises a nucleic acid guided DNA binding domain, for example, a CRISPR-Cas protein, for example, a Cas9 nickase, a Cpf1 nickase, or another CRISPR-Cas nuclease. In some embodiments, the polypeptide domain having DNA polymerase activity comprises a template-dependent DNA polymerase, for example, a DNA-dependent DNA polymerase or an RNA-dependent DNA polymerase. In some embodiments, the DNA polymerase is a reverse transcriptase. In some embodiments, the prime editor comprises additional polypeptides or polypeptide domains involved in prime editing, for example, a polypeptide domain having 5′ endonuclease activity, e.g., a 5′ endogenous DNA flap endonucleases (e.g., FEN1), for helping to drive the prime editing process towards the edited product formation. In some embodiments, the prime editor further comprises an RNA-protein recruitment polypeptide, for example, a MS2 coat protein.

In some embodiments, the DNA binding domain and a DNA polymerase domain that are derived from different species. In some embodiments, a prime editor comprises a Cas polypeptide and a reverse transcriptase polypeptide that are derived from different species. For example, a prime editor may comprise a S. pyogenes Cas9 polypeptide and a Moloney murine leukemia virus (M-MLV) reverse transcriptase polypeptide.

In some embodiments, polypeptide domains of a prime editor may be fused or linked by a peptide linker to form a fusion protein. In other embodiments, a prime editor comprises one or more polypeptide domains provided in trans as separate proteins, which are capable of being associated to each other through non-peptide linkages or through aptamers or recruitment sequences.

### CRISPR On and Off

Over the past decade, the CRISPR-Cas9 gene editing system has revolutionized genetic engineering, allowing scientists to make targeted changes to organisms' DNA. While the system could potentially be useful in treating a variety of diseases, CRISPR-Cas9 editing involves cutting DNA strands, leading to permanent changes to the cell's genetic material. Researchers describe a new gene editing technology called CRISPRoff that allows researchers to control gene expression with high specificity while leaving the sequence of the DNA unchanged. The method is stable enough to be inherited through hundreds of cell divisions, and is also fully reversible.

The classic CRISPR-Cas9 system uses a DNA-cutting protein called Cas9 found in bacterial immune systems. The system can be targeted to specific genes in human cells using a single guide RNA, where the Cas9 proteins create tiny breaks in the DNA strand. Then the cell's existing repair machinery patches up the holes. Because these methods alter the underlying DNA sequence, they are permanent. Moreover, their reliance on “in-house” cellular repair mechanisms means it is hard to limit the outcome to a single desired change. There is thus a need for a different kind of gene editor—one that didn't alter the DNA sequences themselves, but instead changed the way they were read in the cell. This sort of modification is what scientists call “epigenetic”—genes may be silenced or activated based on chemical changes to the DNA strand. Problems with a cell's epigenetics are responsible for many human diseases such as Fragile X syndrome and various cancers, and can be passed down through generations.

Epigenetic gene silencing often works through methylation—the addition of chemical tags to certain places in the DNA strand—which causes the DNA to become inaccessible to RNA polymerase, the enzyme which reads the genetic information in the DNA sequence into messenger RNA transcripts, which can ultimately be the blueprints for proteins. To build an epigenetic editor that could mimic natural DNA methylation, the researchers created a tiny protein machine that, guided by small RNAs, can tack methyl groups onto specific spots on the strand. These methylated genes are then “silenced,” or turned off, hence the name CRISPRoff. To investigate the potential of CRISPRoff for practical applications, the scientists have tested the method in induced pluripotent stem cells. These are cells that can turn into countless cell types in the body depending on the cocktail of molecules they are exposed to, and thus are powerful models for studying the development and function of particular cell types.

### Marking

If a mutation associated with a cancer cell is detected, then the mRNA payload would be released. The mRNA payload would contain instructions for the tumor cells to express a custom Universal Cancer Antigen (UCA). In certain embodiments, the UCA can be a non naturally occurring designer protein. mRNA requires a delivery vehicle to protect against nucleases and facilitate cellular uptake and release into the cytoplasm. Efficient in vivo mRNA delivery is critical to achieving therapeutic relevance. Exogenous mRNA must penetrate the barrier of the lipid membrane in order to reach the cytoplasm to be translated to functional protein. mRNA uptake mechanisms seem to be cell type dependent, and the physicochemical properties of the mRNA complexes can profoundly influence cellular delivery and organ distribution. There are two basic approaches for the delivery of mRNA vaccines that have been described to date. First, loading of mRNA into DCs ex vivo, followed by re-infusion of the transfected cells; and second, direct parenteral injection of mRNA with or without a carrier. Ex vivo DC loading allows precise control of the cellular target, transfection efficiency and other cellular conditions, but as a form of cell therapy, it is an expensive and labor-intensive approach to vaccination. Direct injection of mRNA is comparatively rapid and cost-effective, but it does not yet allow precise and efficient cell-type-specific delivery, although there has been recent progress in this regard. Both of these approaches have been explored in a variety of forms. See, e.g., Pardi, infra, (2018).

### mRNA Delivery

Lipid nanoparticles (LNPs) are an example of a leading non-viral delivery system for mRNA that effectively solves several challenges facing mRNA delivery including protection from nucleases, delivery and release into the cytoplasm, laborious and time-consuming viral packaging and limitations in nucleic acid length with viral vectors. Although less clinically advanced than LNPs, polymers offer similar advantages to lipids and effectively deliver mRNA. Cationic polymers condense nucleic acids into complexes called polyplexes that have various shapes and sizes and can be taken up into cells by endocytosis. The mechanisms by which polyplexes escape from endosomes are uncertain; one possible mechanism is that proton buffering by the polymer leads to osmotic swelling and rupture of the endosomes—the proton sponge hypothesis. Polyethylenimine is the most widely studied polymer for nucleic acid delivery. Although its efficacy is excellent, its application is limited by its toxicity owing to its high charge density. Use of a low molecular weight form, incorporation of PEG into the formulation, conjugation to cyclodextrin and disulfide linkage can mitigate the toxicity of polyethylenimine.

Additionally, several alternative biodegradable polymers have been developed that are less toxic. Poly(β-amino ester)s, for example, excel at mRNA delivery, especially to the lung. Because they are easily synthesized by the Michael reaction, large poly(β-amino ester) libraries have been created that facilitate structure-function studies. Similar to poly(β-amino ester)s, poly(amidoamine)s are biodegradable polymers that are synthesized by the Michael reaction and allow facile modifications to their core and periphery. Poly(amidoamine)s form hyperbranched tree-like spherical dendrimers that efficiently form mRNA complexes owing to the high amine density on their periphery. Although charge density is favourable for mRNA complexation, excessive charge can cause toxicity and serum aggregation. Fortunately, these issues can be mitigated by introducing disulfide linkages or incorporating PEG in the dendrimer core. Like the ionizable lipids in LNPs, pH-responsive polymers have also been used for mRNA delivery. Poly(aspartamide)s conjugated to ionizable aminoethylene side chains are protonated at the acidic pH inside endosomes, facilitating RNA delivery. The hydrophobicity and length of the side chain influence poly(aspartamide) protonation and delivery efficacy. For example, PEGylated poly(aspartamide) with an ethylenediamine side chain delivers mRNA to liver, brain, spinal cord, knee joint and olfactory nerves. In addition to poly(aspartamide)s, pH-responsive charge-altering releasable transporters have gained attention owing to their unique mRNA delivery mechanism. Instead of protonating inside endosomes, these charge-altering releasable transporters self-degrade into neutral, non-toxic by-products at cytosolic pH, leading to rapid release of the mRNA into the cytoplasm.

In addition to lipid and polymer-based vehicles, peptides can also deliver mRNA into cells, thanks to the cationic or amphipathic amine groups (for example, arginine) in their backbone and side chains that electrostatically bind to mRNA and form nanocomplexes. For example, a fusogenic cell-penetrating peptide containing repetitive arginine-alanine-leucine-alanine (RALA) motifs changes conformation at endosomal pH, facilitating pore formation in the membrane and endosomal escape. RALA delivers mRNA to dendritic cells (professional antigen-presenting cells of the immune system) to elicit T cell-mediated immunity. There is also a commercially available cell-penetrating peptide, PepFect14, that delivered mRNA to ovarian cancer cells in a mouse xenograft model. Arginine-rich protamine peptides (of about 4 kDa), which are positively charged at neutral pH, can also condense mRNA and facilitate its delivery. Protamine complexed with mRNA activates Toll-like receptor (TLR7, TLR8) pathways that recognize single-stranded mRNA; thus, it can act as an adjuvant for vaccine or immunotherapy applications. Finally, squalene-based cationic nanoemulsions also deliver mRNA. These nanoemulsions consist of an oily squalene core stabilized by a lipid shell that adsorbs mRNA onto its surface. Some squalene formulations act as adjuvants in influenza vaccines.

The majority of mRNA vaccines are administered as a bolus injection into the skin, muscle or subcutaneous space, where they are taken up by immune or non-immune cells and translated into antigens that are displayed to T and B cells. Both the mRNA and the delivery vehicle enhance the immunogenicity and efficacy of mRNA vaccines.

Recently, mRNA vaccines have generated significant interest to complement or even replace traditional vaccines due to a number of important attributes that they possess. Although subunit vaccines have been used successfully to elicit humoral immunity against a wide variety of pathogens, they fail to induce cellular immunity which is required to eradicate the intracellular pathogen reservoir of many chronic diseases, including viral infections such as HIV or hepatitis C. Live-attenuated vaccines are the most potent in activating both arms of the adaptive immune system—cellular and humoral immunity. However, these vaccines exhibit considerable safety drawbacks. Indeed, attenuated pathogens have the very rare potential to revert to a pathogenic form and cause disease. This is of special concern in immune deficient individuals, or in immunosuppressed patients, where guidelines generally recommend that no live-attenuated vaccines should be administered. Subunit vaccines have been developed as a safer alternative, while recognizing that they are less efficient and often require adjuvants. Andreas M Reichmuth, Matthias A Oberli, Ana Jaklenec, Robert Langer, Daniel Blankschtein, Ther Deliv. 2016 May; 7 (5): 319-334. Published online 2016 Apr. 14. doi: 10.4155/tde-2016-0006.

DNA and mRNA vaccines share many similarities, where the main difference between the two vaccines is the target location for the delivery of the oligonucleotides. DNA therapeutics have to reach the nucleus, while for mRNA therapeutics, the cytosol is the target. As a result, mRNA therapeutics are easier to deliver because they do not require crossing the nuclear membrane. In addition, even if mRNA reaches the nucleus, it does not integrate itself or alters the genome. Although recombination among single-stranded RNA is rarely possible, cytosolic mRNA has no interaction with the genome. Moreover, mRNA essentially represents the minimal genetic information, and is only transiently expressed until the mRNA has been degraded. mRNA can encode multiple proteins possessing very different chemical and physical properties, while leaving its physiochemical properties largely unaffected. Accordingly, mRNA provides the technological basis to deliver a wide variety of antigens, modulators and cell-signaling factors in a single molecule. Simultaneously, mRNA exhibits self-adjuvating properties in that it binds to pattern-recognition receptors like TLR7 that promote cellular immunity. Finally, mRNA synthesis and purification are fast, easy and low cost when compared with other vaccines. Reichmuth (2016)

Unprotected mRNA delivered by itself is unsuitable for broad therapeutic applications, and was therefore ignored by the pharmaceutical industry for a long time. It was the development of RNA interference and its tremendous therapeutic potential that triggered intense efforts toward stabilization of RNA in vivo. Several strategies have been developed for RNA delivery, including RNA-conjugates, modified RNA, viral vectors and microparticles and nanoparticles. While linking RNA to molecules offers some level of protection against degradation, it can promote binding to serum proteins and subsequent aggregation that can lead to vascular blockage. Viral vectors were the obvious choice for delivery, because viruses have naturally evolved to become highly efficient at nucleic-acid delivery. However, several limitations are generally associated with these vectors, including immunogenicity, carcinogenesis, broad tropism packaging capacity and production difficulties. In contrast to viral analogues, nonviral vectors exhibit significantly reduced transfection efficiency but tend to have lower immunogenicity than viruses and patients do not have pre-existing immunity against the nonviral vector. Furthermore, nonviral vectors, whose sizes are larger than those of viruses, have the potential to carry larger genetic payloads, while at the same time being simple to synthesize. With the development of new materials and preparation techniques, as well as a better understanding of the mechanisms involved, nonviral vectors are becoming the preferred vehicle to deliver mRNA. The most common technologies use lipids, polymers, followed by peptides and inorganic nanoparticle. Reichmuth (2016)

Independent of the materials or technologies used, ‘good’ nonviral vectors should: efficiently bind and condense RNA, protect against degradation in the extracellular space and localize the payload at the membrane of the desired target cell, followed by cellular uptake and endosomal escape into the cytosol. The most important targets for mRNA vaccines are professional antigen presenting cells (APCs), with dendritic cells (DCs) likely being the most relevant cell type. Indeed, DCs play a critical role in antigen processing and presentation to elicit an immune response against specific antigens. The transfected DCs express the mRNA-encoded antigen in the native form. The antigens are subsequently processed by the proteasome, and the generated peptide epitopes enter the endoplasmic reticulum where they are loaded onto major histocompatibility complex (MHC) class I molecules.

### Vaccination

An antibody is a protein complex (also referred to as immunoglobulin) uniquely designed to look for antigens, a specific structure found on a foreign virus or particle. When an antibody binds to the antigen, it serves as a flag to attract disease-fighting molecules or as a trigger that promotes cell destruction by other immune system processes. The difficulty is that cancer cells may outpace the immune system, avoid detection, or block immune system activity.

The main direct mechanism by which many antibodies induce tumor cell death is the blockade of growth factor receptor signaling. Pro-tumor growth and survival signaling is perturbed when mAbs bind their target growth factor receptors and manipulate their activation state or block ligand binding. For example, epidermal growth factor receptor (EGFR) is overexpressed by many different cancers and signaling via EGFR leads to tumor cell proliferation, migration, and invasion. Cetuximab, for example, which is an anti-EGFR mAb, induces apoptosis in tumor cells by blocking ligand binding and receptor dimerization. Human epidermal growth factor receptor 2 (HER2) is a tyrosine kinase receptor that is overexpressed in many cancers but primarily ovarian and breast carcinomas. It is distinct from EGFR in that it has no known ligand and instead hetero-dimerizes with other growth factor receptors to enhance their activation. Antibodies targeting HER2 therefore achieve signaling perturbation by inhibiting hetero-dimerization and internalization. Trastuzumab was the first FDA approved anti-HER2 mAb and remains a vital component of treatments for HER2-amplified breast cancer. Indirect mechanisms of action of mAbs require the engagement of components of the host immune system and are CDC, antibody-dependent cellular phagocytosis (ADCP), and ADCC. Most targeted mAbs are able to activate the complement system. For instance, rituximab depends in part on CDC for its in vivo efficacy. In a preclinical model, rituximab anti-tumor effects were completely abolished by knockout of the complement cascade component C1q. The importance of CDC in mAb therapy is further supported by the fact that genetic polymorphisms in the C1qA gene correlate with clinical response to rituximab in patients with follicular lymphoma. Likewise, optimization of CDC via antibody engineering can enhance anti-tumor activity. For example, the anti-CD20 mAb ofatumumab, which mediates amplified CDC, demonstrated greater efficacy than rituximab in a clinical trial of chronic lymphocytic leukemia (CLL) patients. ADCP occurs when FcγRI expressed on cells such as macrophages binds to IgG1 or IgG3 mAbs that have opsonized a tumor cell. There have been very limited studies of ADCP; however, there is some evidence that ADCP plays an important role in destruction of circulating tumor cells following mAb therapy. First described in 1965 by Erna Möeller, ADCC has since been established as an immune mechanism where target cells become opsonized by antibodies which then recruits effector cells to induce target cell death by non-phagocytic mechanisms. Antibodies act as bridges between by binding to antigens on the target cell surface via their Fab portions and linking the effector cells via their Fc portions. While IgG, IgA, and IgE can all mediate ADCC, IgG1 is the most relevant subclass for anti-cancer therapeutic antibodies. Effector cells must express FcR that will bind the antibody in order to facilitate ADCC. Each class of antibody has a corresponding class of FcR such as FcγR, which binds IgG, and FcαR, which binds IgA. FcγR is the most relevant class to ADCC of tumor cells and encompasses both the activating FcγRI (CD64), FcγRIIA (CD32A), FcγRIIIA (CD16A), and inhibitory FcγRIIB (CD32B) receptors. When an activating FcγR on an effector cell binds the Fc region of an antibody receptor crosslinking and downstream signal propagation occurs. NK cells are the main effector type that mediate ADCC; however other myeloid types such as monocytes, macrophages, neutrophils, cosinophils, and dendritic cells are also capable. Effector cells induce target cell death via cytotoxic granule release, Fas signaling, and initiation of reactive oxygen species. While several myeloid cell types have been demonstrated to mediate ADCC during immunotherapy, the clinical efficacy of most targeted mAbs is mainly NK cell dependent.

A UCA-antibody can be developed to target those cells with UCA expression (identified cancer cells) and induce an immune response. In particular, the UCA antibody can bind to, and inhibit the function of, proteins expressed by cancer cells. The UCA antibody can induce an immune response that can cause cell death (apoptosis). Immunotherapy has been used to treat several types of cancers including for example, bladder cancer, brain cancer (brain tumor), breast cancer, cervical cancer and ovarian cancer, colorectal (colon) cancer, head and neck cancer, kidney cancer, liver cancer and lung cancer and leukemia. There are several main types of immunotherapy that may be applied with the claimed vaccine platform.

The antibody can be a monoclonal antibody (mAbs or MoAbs)—man-made versions of immune system proteins designed to attack a very specific part of a cancer cell (in this case, for example, the UCA). Monoclonal antibodies are laboratory-produced molecules engineered to serve as substitute antibodies that can restore, enhance or mimic the immune system's attack on cancer cells. They are designed to bind to antigens that are generally more numerous on the surface of cancer cells than healthy cells Monoclonal antibodies are designed to function in different ways. A particular drug may actually function by more than one means. The role of the drug in helping the immune system may include the following:

Flagging cancer cells. Some immune system cells depend on antibodies to locate the target of an attack. Cancer cells that are coated in monoclonal antibodies may be more easily detected and targeted for destruction.

Triggering cell-membrane destruction. Some monoclonal antibodies can trigger an immune system response that can destroy the outer wall (membrane) of a cancer cell.

Blocking cell growth. Some monoclonal antibodies block the connection between a cancer cell and proteins that promote cell growth-an activity that is necessary for tumor growth and survival.

Preventing blood vessel growth. In order for a cancerous tumor to grow and survive, it needs a blood supply. Some monoclonal antibody drugs block protein-cell interactions necessary for the development of new blood vessels.

Blocking immune system inhibitors. Certain proteins that bind to immune system cells are regulators that prevent overactivity of the system. Monoclonal antibodies that bind to these immune system cells give the cancer-fighting cells an opportunity to work with less inhibition.

Directly attacking cancer cells. Certain monoclonal antibodies may attack the cell more directly, even though they were designed for another purpose. When some of these antibodies attach to a cell, a series of events inside the cell may cause it to self-destruct.

Delivering radiation treatment. Because of a monoclonal antibody's ability to connect with a cancer cell, the antibody can be engineered as a delivery vehicle for other treatments. When a monoclonal antibody is attached to a small radioactive particle, it transports the radiation treatment directly to cancer cells and may minimize the effect of radiation on healthy cells. This variation of standard radiation therapy for cancer is called radio immunotherapy.

Delivering chemotherapy. Similarly, some monoclonal antibodies are attached to a chemotherapeutic drug in order to deliver the treatment directly to the cancer cells while avoiding healthy cells.

Binding cancer and immune cells. Some drugs combine two monoclonal antibodies, one that attaches to a cancer cell and one that attaches to a specific immune system cell. This connection may promote immune system attacks on the cancer cells.

In certain embodiments, the antibody can be a bi-specific antibody that binds T-cells to ensure a more precise immune response can be used. In certain embodiments, the antibody may be used as a checkpoint inhibitor, or a drug that takes the ‘brakes’ off the immune system, which helps it recognize and attack cancer cells. In other embodiments, the antibody can be used as a part of a chimeric antigen receptor (CAR) T-cell therapy. This therapy takes some T-cells from a patient's blood, mixes them with a special virus that makes the T-cells learn how to attach to tumor cells, and then gives the cells back to the patient so they can find, attach to, and kill the cancer.

In yet other embodiments, the antibody can be used to modulate, activate or inhibit cytokines (small proteins that carry messages between cells) to stimulate the immune cells to attack cancer. The cytokines can include interferon or interleukin proteins.

In yet other embodiments, the antibody can also be used as a general immunomodulatory drug that generally boosts parts of the immune system to treat certain types of cancer.

### EXAMPLES

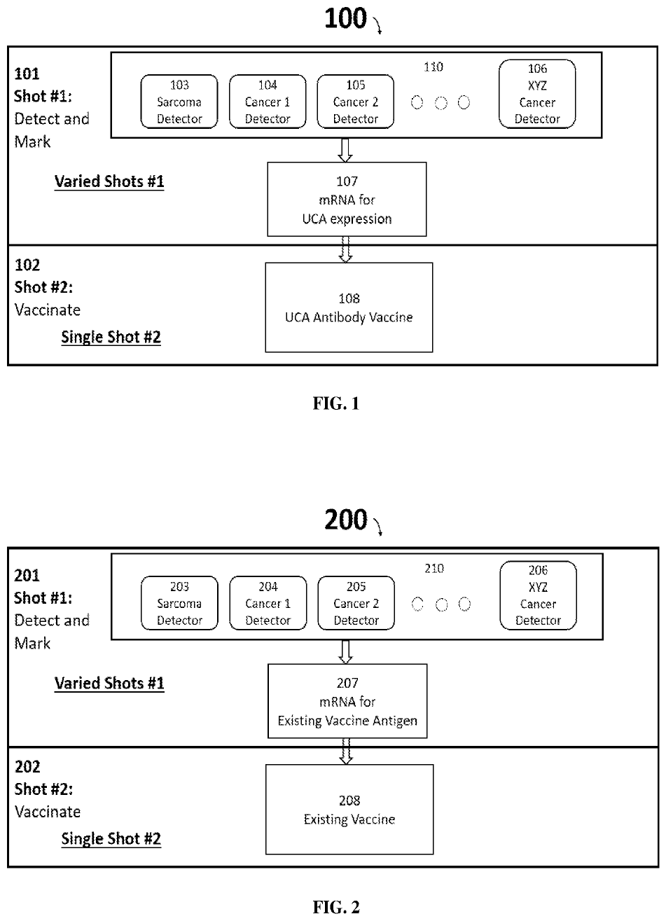

Referring to FIG. 1, the vaccine platform 100 can include a CRISPR-based in body DNA/RNA detector 110. This can include for example, a sarcoma detector 103, a first cancer detector 104 (to detect a first specific cancer type), a second cancer detector 105 (to detect a second specific cancer type), or a general cancer detector 106, not specific to any particular cancer. The initial step of the platform is a detect and mark step, which can include administering varied shots (e.g. parenterally) that release mRNA 107 containing instructions for the expression of UCA expression in the targeted cells. The second step is the vaccination step, which can include administering a second shot containing a UCA antibody 108 that targets the cells expressing the UCA.

Referring to FIG. 2, the vaccine platform 200 can include a CRISPR-based in body DNA/RNA detector 210. This can include for example, a sarcoma detector 203, a first cancer detector 204 (to detect a first specific cancer type), a second cancer detector 205 (to detect a second specific cancer type), or a general cancer detector 206, not specific to any particular cancer. The initial step of the platform is a detect and mark step, which can include administering varied shots (e.g. parenterally) that release mRNA for an existing vaccine antigen 207. The second step is the vaccination step, which can include administering an existing vaccine 208 that targets the cells expressing the vaccine antigen 207.

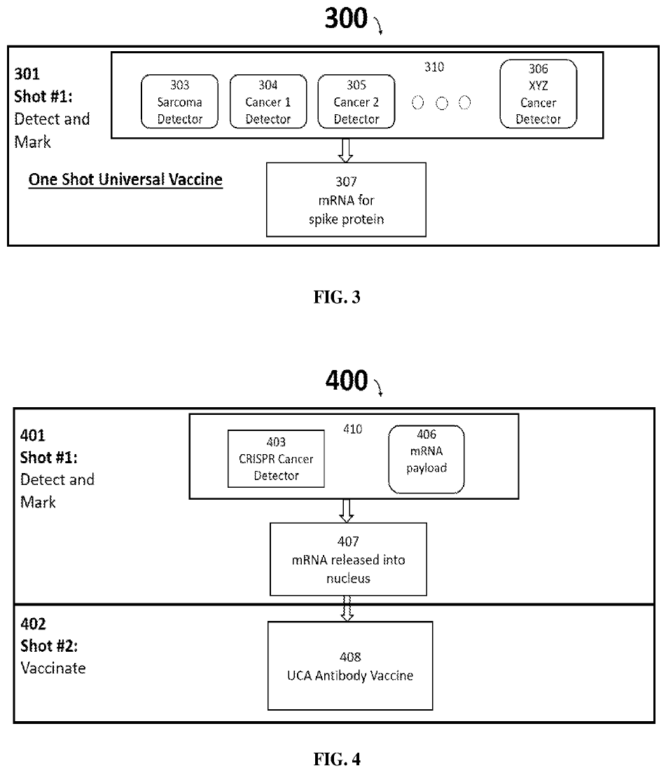

Referring to FIG. 3, the vaccine platform 300 can be a one-shot universal cancer vaccine platform. The premise of this platform is that with the global rollout of certain vaccines (e.g., COVID-19 vaccine), billions of vaccinated people around the world have immune systems that are already producing antibodies to detect and kill cells/virus with certain antigens (e.g., the SARS-CoV-2 spike protein or COVID antigen). In this embodiment, the therapy would be administered to a subject that has been previously vaccinated against the target antigen (e.g. COVID-19 antigen). In these patients, a cancer cell can targeted and made to express the same spike protein, effectively tricking the body's immune system into thinking that the cancer cell is an antigen (e.g., COVID-19 antigen) and attack it accordingly. This platform can include for example, a sarcoma detector 303, a first cancer detector 304 (to detect a first specific cancer type), a second cancer detector 305 (to detect a second specific cancer type), or a general cancer detector 306, not specific to any particular cancer. The method can include administering a shots (e.g. parenterally) that releases mRNA that induces expression of a known target antigen 307 (e.g. spike protein such as a COVID spike protein). The antibodies that are in the subject's body can then mobilize to attack the cancer cells that have the target antigen (e.g. spike protein) and then mount an immune response.

Referring to FIG. 4, the vaccine platform 400 can be a cancer vaccine platform in which the CRISPR detector 403 and the mRNA payload 406 are provided in a single composition 410 for injection in a first step 401 for detection and marking of the target mutation (e.g. cancer marker). In certain embodiments, the mRNA and CRISPR detector can enter the cell nucleus together. Upon a positive match of the target genetic sequence (e.g. a detected cancer mutation), the mRNA payload is released for UCA protein production. In a subsequent step 402, a subsequent shot can deliver a UCA antibody vaccine 408 to the target antigen and then mount an immune response.

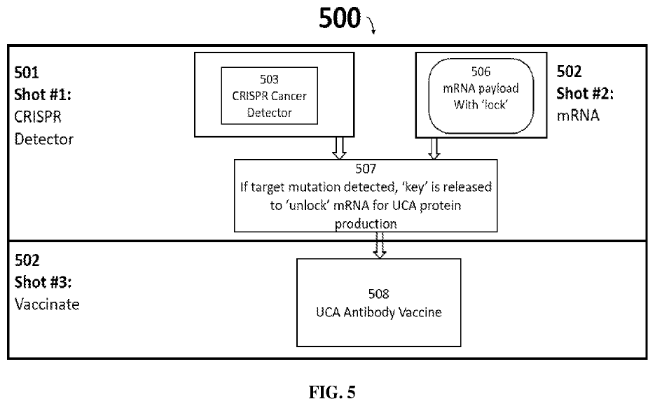

Referring to FIG. 5, the vaccine platform 500 can be a cancer vaccine platform in which the CRISPR detector 503 and the mRNA payload 506 are provided in separate injections. The first “shot” is a CRISPR-detector-with-key that is a monolithic compound that enters the nucleus for genetic detection. The second “shot” is an mRNA only compound with a “lock.” If the target genetic mutation (e.g. for a specific cancer) is detected, then the “key” (a chemical compound) 507 is released to unlock the mRNA compound for translation and antigen protein production. Subsequently, a third shot 508 can deliver a UCA antibody vaccine to the target antigen and then mount an immune response as previously described.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

What is claimed is:

1. A method for targeting cancer-associated mutations comprising:
identifying a nucleic acid mutation associated with a specific cancer,
detecting the nucleic acid mutation using a CRISPR detector and an mRNA payload, upon detection, releasing the mRNA payload into a target cell, the mRNA payload containing instructions for the target cell to express a universal cancer antigen (UCA);
allowing the target cell to express a universal cancer antigen; and
allowing a UCA antibody designed to target those cells expressing the UCA.

2. The method of claim 1, wherein the universal cancer antigen is a non-naturally occurring protein.

3. The method of claim 1, wherein the nucleic acid is DNA.

4. The method of claim 1, wherein the nucleic acid is RNA.

5. The method of claim 1, wherein the antibody induces cell death of the target cell.

6. The method of claim 1, wherein the antibody reduces tumorigenesis.

7. The method of claim 1, wherein the antibody reduces angiogenesis.

8. The method of claim 1, wherein the antibody activates an immune response.

9. The method of claim 8, wherein the antibody activates an effector cell response.

10. The method of claim 8, wherein the antibody activates a memory cell response.

11. The method of claim 1, wherein the antibody modulates a cytokine response.

12. The method of claim 11, wherein the antibody modulates an interferon response.

13. The method of claim 11, wherein the antibody modulates an interleukin response.

14. The method of claim 1, wherein the antibody is a bi-specific antibody that binds T-cells to ensure a more precise immune response.

15. The method of claim 1, further comprising administering an adjuvant.

16. The method of claim 1, wherein the CRISPR detector and mRNA payload are provided together in a single shot

17. The method of claim 1, wherein the CRISPR detector and mRNA payload result in expression of a universal cancer antigen that is targeted by a subject's existing antibodies.

18. The method of claim 17, wherein the universal cancer antigen is targeted by an antibody provided in a vaccine.

19. The method of claim 1, wherein the CRISPR detector and mRNA payload are provided in separate shots.

20. The method of claim 18, wherein the shots are provided simultaneously.

21. The method of claim 18, wherein the shots are provided sequentially.

22. The composition for treating cancer comprising:
a CRISPR detector and
an mRNA payload containing instructions for the tumor cell to express a universal cancer (UCA) antigen and
a UCA antibody designed to target those cells with UCA expression.

23. A method for targeting cancer-associated mutations comprising:
identifying a nucleic acid mutation associated with a specific cancer,
detecting the nucleic acid mutation using a CRISPR detector and a payload containing genetic material,
upon detection, releasing the mRNA payload into a target cell, the payload containing instructions for the target cell to express a universal cancer antigen (UCA);
allowing the target cell to express a universal cancer antigen; and
allowing a UCA antibody designed to target those cells expressing the UCA.

24. The method of claim 23, wherein the genetic material is non-replicating mRNA.

25. The method of claim 23, wherein the genetic material is virally derived mRNA.

26. The method of claim 23, wherein the genetic material is self-amplifying RNA.
